(19) United States
(12) Patent Application Publication
(10) Pub. No.: US 2003/0190345 A1
(43) Pub. Date: Oct. 9, 2003

(54) Plaster preparation

(75) Inventors: Gunter Cordes, Leichlingen (DE); Ulrike Vollmer, Dormagen (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/370,311

(22) Filed: Feb. 13, 2003

Related U.S. Application Data
(63) Continuation of application No. PCT/EP01/05603, filed on May 16, 2001.

(30) Foreign Application Priority Data
May 17, 2000 (DE) 100 24 396.7

(51) Int. Cl. A61K 9/70

(52) U.S. Cl. 424/443

(57) ABSTRACT

The invention relates to a coloured plaster cover that can be used, e.g., with children, in the case of irritations (formications, burns and itches) resulting from contact with stinging nettles or similar irritations.

### CROSS-REFERENCE TO RELATED APPLICATIONS

This is a continuation application of PCT/EP01/05603, filed May 16, 2001, which is incorporated herein by reference in its entirety, and also claims the benefit of German Priority Application No. 100 24 396.7, filed May 17, 2000.

### FIELD OF THE INVENTION

The invention relates to plaster preparations that can be used in the case of irritations (formications, burns and itches) resulting from contact with stinging nettles or similar irritations.

### BACKGROUND OF THE INVENTION

Skin irritations such as those caused by mosquito bites or contact with stinging nettles or jellyfish are invariably found to be unpleasant and troublesome. The skin irritations described in the present patent specification are all reactions involving slight reddening, weals, blisters but not pathological states such as high-degree sensitisation resulting in anaphylactic shock or the secondary formation of granulomas in the clinical picture of prurigo. The skin irritations referred to here, which are to be treated with the product according to the invention, chiefly occur after time spent outdoors, for example as occurring during children's play in woods, fields or at the edge of water but also during summer holidays or in hot resorts, or while playing sports although they may also be acquired otherwise. The skin irritations described above should be interpreted as follows in medical terms.

According to textbooks (e.g., Jung, Dermatologie, 1991), minor irritation at the nociceptors of the skin produces itching while more serious irritation causes pain. Itching may also be a reaction to chemical, mechanical or thermal stimuli. This results in weals and/or urticaria through the effect of chemical substances such as from insect bites (e.g., histamine from mosquito bites, apamin from bee stings, stinging nettles etc.), allergic immune reactions to chemical substances; the effect of pressure and/or rubbing (e.g. with a paper clip or a wooden spatula, the rubbing of fine hairs), from heat (sport, heated pads) and from light (sunburn). An itchy weal and/or urticaria shows all the signs of irritation such as reddening, swelling, heat, “pain” or itching and is due to the cutaneous release of histamine. Itching has not been fully explained in medical terms and always involves a subjective, in some cases psychological, element.

There have been a number of reliable remedies for itching on the market for some years. Here use is generally made of cooling gels and sticks which contain antihistamines in addition to cooling through the evaporation of alcohol or water. Use is also made of isoprenaline in powder or cream form, something which like the counter-irritant crotamiton is not recommended in the long term due to skin irritation, apart from the fact that the effect cannot be verified or explained in rational terms. The disadvantage of these topical formulations is their brief action, something which means that the application has to be constantly renewed. In addition, the insufficient length of action may result in the bites or stings becoming infected due to scratching. Skin that has become infected or damaged through scratching is much slower to heal and may scar. Then there are treatments involving oral antihistamines but they are not the preferred choice for such discomfort, which cannot be described as being pathological in nature, and have the side effect of impairing the ability to react due to sedation.

The patent specification DE 19833177 A1 describes how the application of a plaster can be very effective against the consequences of insect bites or contact with stinging nettles if a certain composition is used for the base and inactive ingredients and if it is ensured that the substances used have prolonged contact with the part of the skin injured and/or concerned and scratching is simultaneously prevented. The plaster described therein contains a combination of menthol and a local anaesthetic of the ester type (benzocaine).

### SUMMARY OF THE INVENTION

The invention relates to plaster preparations that can be used in the case of irritations (formications, burns and itches) resulting from contact with stinging nettles or similar irritations. In particular, the plaster preparations include the substances menthol, thesit and tincture of plantain. The preparation diminishes the irritant effect, which manifests itself particularly in formications, itches and burns, following contact with stinging nettles and insect bites.

### DETAILED DESCRIPTION OF THE INVENTION

Surprisingly, a biological double-blind placebo-controlled laboratory study, which tested various formulations using standardised mosquito bites (Aedes aegypti), has shown that the existence of the local anaesthetic of the ester type (benzocaine base) is not necessary. It has been discovered that the local anaesthetic of the ester type can be replaced by adding 1% tincture of plantain to achieve the same effect. In the biological test involving Aedes aegypti mosquitoes (bred pathogen-free by the University of Bonn/Parasitology) 2-4 insects were positioned per subject on the lower arm of 4 volunteers using a glass cylinder until at least 1-2 bites had occurred on each underarm. As soon as the subject experienced itching, the bites were measured and covered with plasters on both lower arms (blind randomised test). The subjects were then questioned about their perceptions 10, 20 and 30 minutes after application. Two hours after application the plaster was removed and the size of the bite measured. The following formulations were tested.

| Formulation | Active ingredients (level in adhesive matrix) |
|---|---|
| A | Menthol (5%), benzocaine (8%), polidocanol (8%) |
| B | Menthol (5%), extract of plantain (1%), polidocanol (8%) |
| C | Menthol (5%) |
| D | Placebo |

The following result was observed:

| Formulation | Itching | Reddening |
|---|---|---|
| A | Abated after 10 min, no itching | Reduction in diameter by |
| | after 20 min | approx. 0.5 cm |
| B | No itching after 20 min | Reduction in diameter by |
| | | approx. 0.5 cm |
| C | Abated after 30 min | Partial reduction in |
| | | diameter by approx. 0.5 |
| | | cm |
| D | Unchanged after 30 min | Reduction in diameter by |
| | | approx. 0.5 cm |

As local anaesthetics are also known to be capable of causing contact allergies and the addition of benzocaine is not permitted for subsequent distribution as a cosmetic product, the formulation was developed further without a local anaesthetic specifically for use with children.

In children, covering the irritation with a plaster has a pronounced psychological effect. For this purpose the cover must appeal to children in terms of appearance. This can be achieved for example by printing on popular cartoon figures (Sesame Street, Pooh Bear, Teletubbies, etc.) that can be applied to the backing foil using the scatter printing process. Another option is plain-colour inking. As this would necessitate ordering from the foil supplier, checking and stocking several backing foils in different colours, a logistically and economically more favourable variant was developed whereby the plaster formulation itself can be coloured by adding non-toxic and cosmetically suitable dyes. The use of a transparent backing foil then produces coloured plaster covers which appeal to children and can be used to treat skin irritations after insect bites, contact with stinging nettles etc. Here it is important to use dyes that are physiologically harmless, dissolve in the plaster formulation and do not leave any discolouration on the skin when in contact with it. Specialists can then produce plasters in virtually any colour desired (yellow, orange, red, turquoise, green, etc.). Amaranth is, for example, not suitable as it does not dissolve in solvent-based acrylate adhesive bases, or methylene blue as it dyes the skin blue when in contact with it. It may be useful to also add titanium dioxide besides the dye to intensify the colour of the transparent plaster.

The present invention will be further described by the following non-limiting examples that demonstrate formulations according to the invention.

### EXAMPLE 1

To produce 1800 cm² of a yellow plaster laminate according to the invention on a laboratory scale 1.385 g menthol, 1.440 g thesit and 4.5 g tincture of plantain 4%, 43.927 g Durotak® 87-2852 and 2.3 ml ethanol 96% are mixed and added, followed by 323.5 mg beta-carotene, and then homogenised by stirring. The paste is spread with a scraper on a siliconised polyester foil in a wet layer of 400 μm to produce a surface weight of 100 g/m² after drying. After laminating with Cotran # 9720 a bright yellow plaster 4.0 cm² in size contains 1.5 mg menthol.

### EXAMPLE 2

To produce 1800 cm² of a red plaster laminate according to the invention on a laboratory scale 1.385 g menthol, 1.440 g thesit and 4.5 g tincture of plantain 4%, 43.927 g Durotak® 87-2852 and 2.3 ml ethanol 96% are mixed and added, followed by 212.5 mg iron (III) oxide, and then homogenised by stirring. The paste is spread with a scraper on a siliconised polyester foil in a wet layer of 400 μm to produce a surface weight of 100 g/m² after drying. After laminating with Cotran # 9720 a bright red plaster 4.0 cm² in size contains 1.5 mg menthol.

The above examples can of course also be produced without dyes to permit usage by adults, who can then directly see the soothing therapeutic effect of the plaster on the skin irritation through the transparent plaster, and to also offer relief for skin irritations in the facial area as discretely and inconspicuously as possible in cosmetically acceptable terms.

There are also other plaster bases known to the specialist such as hot-melt adhesives, silicones, rubbers and water-based systems although it is necessary to re-adjust the dyes to them in each case.

That which is claimed:

1. A plaster preparation comprising menthol, thesit and tincture of plantain in a plaster matrix.

2. The plaster preparation according to claim 2, wherein the plaster matrix is selected from the group consisting of solvent-based acrylate adhesive bases, hot-melt adhesives, silicones, rubbers and water-based systems.

3. The plaster preparation according to claim 1, wherein the plaster matrix is a hot-melt adhesive from EVA copolymers and aromatic hydrocarbon resins or a solvent acrylate adhesive obtained by means of radical polymerisation from acrylate copolymers.

4. The plaster preparation according to claim 1, wherein the menthol, thesit and tincture of plantain are dissolved in a layer of the plaster matrix having a surface weight between 20 and 200 g/m².

5. The plaster preparation according to claim 4, wherein the layer of the plaster matrix has a surface weight between 80 and 120 g/m².

6. The plaster preparation according to claim 1, wherein the menthol is present in a concentration between 1-10%, the thesit is present in a concentration between 2-10%, and the plantain is present in a concentration between 0.5-5%, on a per weight basis.

7. The plaster preparation according to claim 1, wherein the menthol is present in a concentration between 3-5%, the thesit is present in a concentration of about 8%, and the plantain is present in a concentration of about 1%, on a per weight basis.

8. The plaster preparation according to claim 1, further comprising non-toxic dyes that are authorised for use in cosmetics for colouring the plaster matrix.

9. The plaster preparation according to claim 8, wherein the dyes are soluble in the plaster matrix and do not discolour the skin in contact with the preparation.

10. The plaster preparation according to claim 1, further comprising titanium dioxide to intensify the colour of the plaster matrix.

11. The plaster preparation according to claim 1, further comprising a cover foil attached to the plaster matrix.

12. The plaster preparation according to claim 11, wherein the cover foil bears cartoon figures applied using scatter printing.

13. The plaster preparation according to claim 11, wherein the cover foil is a colourless flexible foil which is made of polyethylene, LDPE, EVA copolymers and polyurethane, preferably of polyethylene.

14. A method of diminishing the irritant effect of formications, itches and burns by applying to the irritated skin a plaster preparation comprising menthol, thesit and tincture of plantain in a plaster matrix.

15. The method according to claim 14, wherein said applying step follows skin contact with stinging nettles or follows an insect bite.

16. The method according to claim 14, wherein said applying step comprises applying a plaster preparation wherein the plaster matrix is selected from the group consisting of solvent-based acrylate adhesive bases, hot-melt adhesives, silicones, rubbers and water-based systems.

17. The method according to claim 14, wherein said applying step comprises applying a plaster preparation wherein the menthol is present in a concentration between 1-10%, the thesit is present in a concentration between 2-10%, and the plantain is present in a concentration between 0.5-5%, on a per weight basis.

18. The method according to claim 14, wherein said applying step comprises applying a plaster preparation that further includes non-toxic dyes that are authorised for use in cosmetics for colouring the plaster matrix.

19. The method according to claim 14, wherein said applying step comprises applying a plaster preparation that further includes titanium dioxide to intensify the colour of the plaster matrix.

20. The method according to claim 14, further comprising the step of attaching a cover foil to the plaster matrix.
